(19) United States
(12) Patent Application Publication
(10) Pub. No.: US 2010/0324562 A1
(43) Pub. Date: Dec. 23, 2010

(54) Broaching punch and method of forming bone tunnels

(75) Inventors: Darren W. Thomsen, Largo, FL (US); Robert Thibodeau, Saint Petersburg, FL (US)

Correspondence Address: GENE WARZECHA;LINVATEC CORPORATION, 11311 CONCEPT BOULEVARD, LARGO, FL, 33773, US

(73) Assignee: LINVATEC CORPORATION

(21) Appl. No.: 12/802,820

(22) Filed: Jun. 15, 2010

Related U.S. Application Data
(60) Provisional application No. 61/268,845, filed on Jun. 17, 2009.

(51) Int. Cl. A61B 17/16

(52) U.S. Cl. 606/80

(57) ABSTRACT

A device and method for forming bone tunnels. The device has a conical tip with a spiral cutting edge intended to be pushed into the bone. After penetrating the bone to the desired depth the device is able to be rotated back out of the tunnel without excessive strain on the wall of the bone tunnel.

The present application is related to and claims the benefit under 35 USC 119(e) of U.S. Provisional Application Ser. No. 61/268,845 filed Jun. 17, 2009.

### BACKGROUND OF THE INVENTION

1. Field of the Invention

The invention relates to devices and methods used to form holes or tunnels in bone. More particularly, the invention relates to broaches and punches used to make bone tunnels or holes intended to receive suture anchors and the like.

2. Description of the Prior Art

The partial or complete tear of ligaments, tendons and other soft tissue from the respective bone attachment sites is a common occurrence due to chronic or acute injury. Surgery may be required to reattach the torn soft tissue to bone. There are many devices on the market for soft tissue repair. Such devices may be threaded suture anchors, where the torn soft tissue is sutured to the anchor and the anchor is secured in the bone with threads, or push-in suture anchors which are placed into pre-formed holes.

Various devices are used to form the holes and enable them to receive the selected suture anchor. (The terms “tunnel” and “hole” are used interchangeably herein). Simple drills have been used to drill holes through the hard cortical layer of bone and into the softer cancellous layer of bone. However, this procedure takes time and sometimes requires expensive equipment. Additionally, drilling removes bone tissue and has the potential for causing bone necrosis through overheating. Bone punches are sometimes used. These are simply pointed shafts having a conical tip which tends to create a hole through the cortex and into the cancellous bone. However, the conical tip tends to expand the cortex around the hole and create stress fractures which weaken the bone, thus comprising its strength. Additionally, when a hole is created with a traditional awl-type punch, the surgeon can have difficulty removing the punch due to the radial compressive forces holding the punch in the bone. The surgeon must either put extra pressure on the bone to pull against it, or wiggle the punch to create sufficient clearance to allow the punch to be removed thereby compromising the size of the hole. Both of these techniques can further fracture the cortical surface and compromise the size, integrity and margin of the hole. These events could result in patient injury or compromise the success of the repair.

With some threaded anchors the surgeon may need to tap threads into a pre-drilled or pre-formed hole. This is helpful in some cases, but does increase the surgery time and increases risk to the patient.

There is a need for a device and method suitable for quickly and easily forming a bone hole or tunnel without the disadvantages of the prior art.

Accordingly it is an object of the present invention to provide a tunnel forming device which decreases damage to the native surrounding bone into which the anchor is inserted.

It is a further object of this invention to provide a tunnel forming device to cut and displace bone tissue as it advances.

### SUMMARY OF THE INVENTION

These and other objects of this invention are achieved by the preferred embodiment disclosed herein. This invention is a broaching punch for forming a hole in bone. The punch comprises
an elongated shaft having an axis, a proximal end and a distal end; a tapered tip situated at the distal end. The tapered tip has a distal-most point on the axis and has a generally spiral broaching surface extending proximally away from the point. The broaching surface comprises a spiral cutting edge formed on the tapered tip, the edge adapted to cut a predetermined size hole as it is pushed into the bone.

In another aspect, this invention is a method of forming a hole in bone comprising providing a broaching punch constructed as described above, pushing the broaching punch a predetermined distance into the bone and turning the broaching punch about its axis to remove it from the bone.

### BRIEF DESCRIPTION OF THE DRAWINGS

FIG. 1 is a front perspective view of the distal tip of a broaching punch constructed in accordance with the principles of this invention.

FIG. 2 is a side elevational view of the distal tip of the instrument shown in FIG. 1.

FIG. 3 is a side elevational view of an alternate embodiment of the instrument in FIG. 1.

### DESCRIPTION OF THE PREFERRED EMBODIMENT

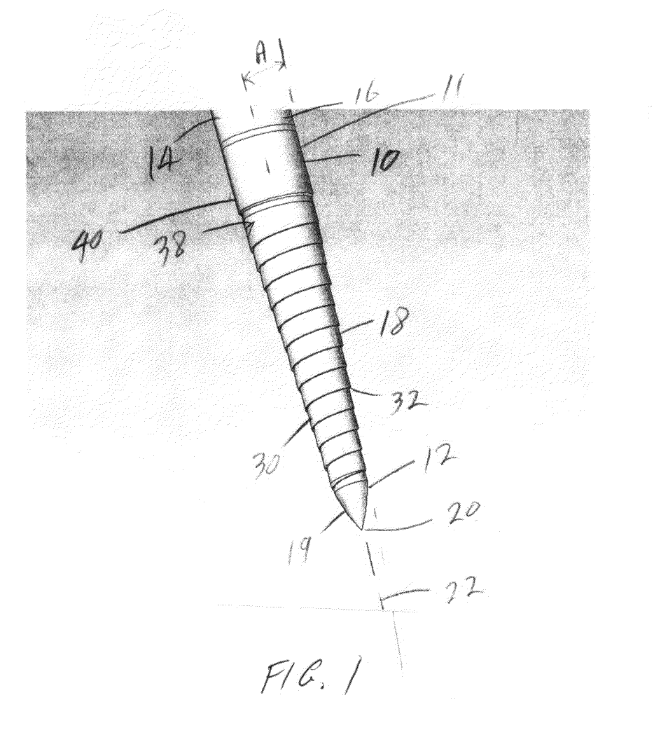

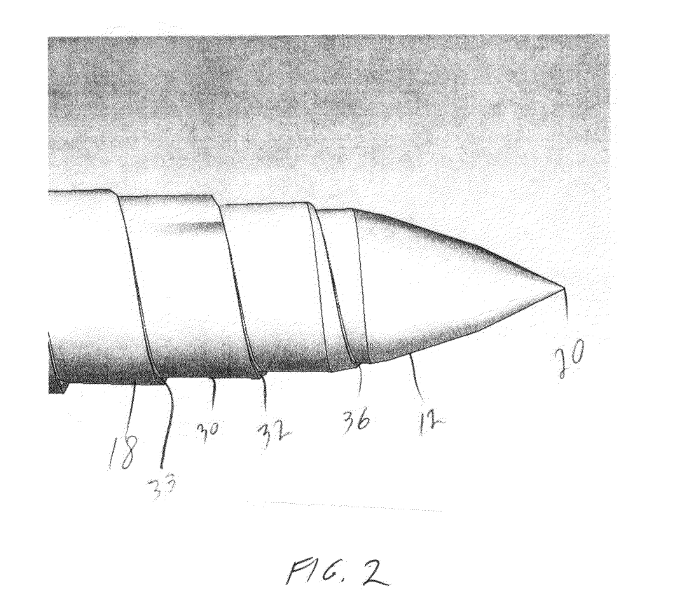

The working, distal end of a broaching punch 10 constructed in accordance with the principles of this invention is shown in FIGS. 1 and 2. Broaching punch 10 has an elongated body 11 with a distal end 12, proximal end 14, intermediate shaft 16 and conical, tapered tip 18. Conical tip 18 terminates in a conical surface 19 having a penetrating distal point 20 which, in the preferred embodiment is sharp enough to be pushed into the cortical layer of bone. The punch may have a mallet impact surface (not shown) at its proximal end or simply a handle.

Conical tip 18 extends proximally at an angle A relative to axis 22 and is provided with a helical broaching surface 30 having a spiral cutting edge 32. Spiral cutting edge 32 faces distally and is a single continuous edge spiraling about axis 22 from a distal point 36 to a proximal point 38. Distal point 36 is smoothly tapered into the body of broaching punch 10 at a spot proximal to the conical surface 19 and tip 20, and proximal point 38 smoothly tapers into the surface of the body of broaching punch 10 at the distal end of conical tip 18. Conical tip 18 abuts at its proximal end to a cylindrical surface 40. Broaching surface 30 and spiral cutting edge 32 may be formed by a complementarily shaped cutting tool translating proximally along body 11 from a point proximal to conical surface 19 while simultaneously retracting radially from axis 22. Broaching surface 30 is generally parallel to axis 22 although it may have other orientations. Cutting edge 32 may simply be a distally facing spiral surface 33 extending radially away from axis 22. Other profiles may be used as well. For example, the radially outermost junction of cutting edge 32 with broaching surface 30 may be chamfered rather than being a right angle. In the preferred embodiment spiral surface 33 extends radially a distance of approximately on the order of 0.003 inches. Conical surface 19 may be formed with any desired tapered angle between point 20 and the transition to spiral cutting edge 32, provided the surface is able to penetrate the bone as intended.

Broaching punch 10 is used by placing tip 22 at a point on a bone where one desires to form a bone hole or tunnel. The axis 22 is aligned with the intended axis of the desired bone hole and broaching punch 10 is moved (e.g., pushed or tapped) into the bone. As the broaching punch 10 advances into the bone, cutting edge 32 cuts the bone which it contacts and pushes the bone debris along with it into the bone hole. This has the incidental and beneficial effect of compacting the bone debris somewhat, thereby enhancing the quality of the wall of the bone hole being formed. When broaching punch 10 has been advanced to the desired depth, that is until some or all of conical tip 18 is within the bone and the hole has the desired diameter, the punch may be withdrawn by simply rotating it counterclockwise about axis 22. If desired, the punch could be advanced into the bone far enough to insert cylindrical surface 40 into the bone.

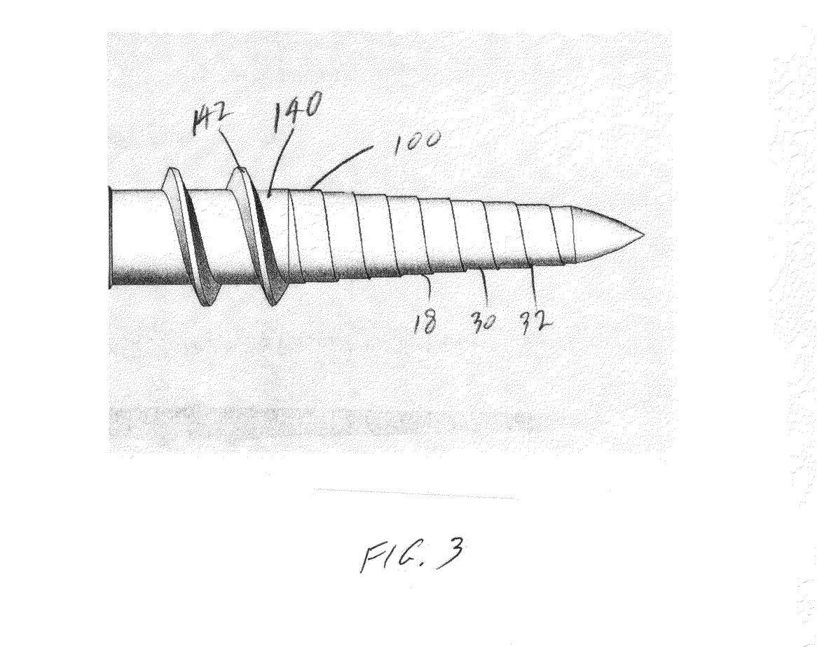

An alternate embodiment of the invention is shown in FIG. 3 as broaching punch 100. The elements in FIG. 3 are identical to like numbered elements in FIGS. 1 and 2. FIG. 3 differs from FIG. 1 in that the surface immediately proximal to conical tip 18 is surface 140 having a tapping thread 142. Broaching punch 100 is used in the same manner as broaching punch 10 except that when the conical tip 18 is completely pushed into the bone, broaching punch 100 is then turned into the bone further in order to tap threads into the cortical (and cancellous) bone at the proximal end of the hole, which threads can then be used by a threaded suture anchor. Broaching punch 100 is removed from the bone by turning it counterclockwise just as with broaching punch 10.

It will be noted that all embodiments of the broaching punch disclosed herein cut the bone upon insertion of the punch into bone, thereby eliminating the stress cracks caused by prior art punches which simply compact cortical bone as the punch is inserted into bone.

The principles of this invention may be used to produce an alternate embodiment of the broaching punch (not shown) in which the conical tip 18 is shorter and transitions into a cylindrical broaching section. That is, rather than using a spiral cutting edge 32 superimposed solely on a conical tip 18, a spiral cutting edge could be superimposed on a cylindrical tip to form a cylindrical hole rather than a tapered hole.

It will be understood by those skilled in the art that numerous improvements and modifications may be made to the preferred embodiment of the invention disclosed herein without departing from the spirit and scope thereof.

What is claimed is:

1. A broaching punch for forming a hole in bone comprising:
an elongated shaft having an axis, a proximal end and a distal end;
a tapered tip situated at said distal end, said tapered tip having a distal-most point on said axis and having a generally spiral broaching surface extending a predetermined distance proximally from a predetermined spot proximal to said point, said broaching surface comprising a cutting edge adapted to cut a predetermined size hole as said broaching punch is pushed into the bone.

2. A broaching punch according to claim 1 wherein said cutting edge is a spiral edge and is radially outwardly extending a distance on the order of 0.003 inches above the broaching surface.

3. A broaching punch according to claim 1 wherein said tapered tip is angled relative to said axis at between 3° and 5°.

4. A broaching punch according to claim 1 further comprising a tapping thread situated proximally to said broaching surface.

5. A method of forming a hole in bone comprising:
providing a broaching punch constructed according to claim 1;
moving said broaching punch a predetermined distance into the bone; and
turning said broaching punch about its axis to remove it from the bone.

6. A method according to claim 5 further comprising the steps of:
using a broaching punch constructed according to claim 4; and
turning said broaching punch after said moving step to tap a thread in said bone.
